(19) United States
(12) Patent Application Publication
(10) Pub. No.: US 2026/0196342 A1
(43) Pub. Date: Jul. 9, 2026

(54) METHOD AND APPARATUS FOR MANAGING MOBILE ANESTHESIA

(71) Applicant: Organano Systems, Inc., Pacific Palisades, CA (US)

(72) Inventors: Richard Pattinson, Pacific Palisades, CA (US); Salman Hussain, Pacific Palisades, CA (US)

(73) Assignee: Organano Systems, Inc., Pacific Palisades, CA (US)

(21) Appl. No.: 19/441,694

(22) Filed: Jan. 6, 2026

Related U.S. Application Data
(60) Provisional application No. 63/742,808, filed on Jan. 7, 2025.

(51) Int. Cl. G16H 40/20; G16H 10/60

(52) U.S. Cl. CPC G16H 40/20; G16H 10/60

(57) ABSTRACT

In an embodiment, a system is provided for coordinating mobile anesthesia services using a case-centric, modular architecture. The system supports scheduling, patient workup, intraoperative documentation, controlled substance compliance, and billing through interoperable modules that exchange case-associated information while operating within distinct functional scopes. The system may assemble case-contextual patient profiles, support real-time intraoperative documentation including voice-based charting with provider confirmation, track controlled substances across acquisition, usage, and waste, and manage billing and payment workflows. An artificial intelligence and machine learning support layer may provide assistive outputs across modules while preserving human review and confirmation. The system maintains traceability and auditability of case-associated information and supports flexible deployment of modules without centralizing clinical decision-making.

This patent application claims priority to U.S. Provisional Patent Application Ser. No. 63/742,808 filed on Jan. 7, 2025, which is incorporated by reference herein in its entirety.

### BACKGROUND OF THE SYSTEM

Many procedures that used to require hospitalization have now become in-office procedures. Even certain surgical procedures are now done in a clinical office instead of in a hospital. Medical care has changed to the point where most surgical procedures no longer require an overnight stay, but allow for same day discharge of the patient. This change helps keep costs down, creates financial incentives for the practitioner, and provides more convenience for the patient. However, in-office surgical procedures require the administration of anesthesia during the procedure.

But, most clinical offices, regardless of specialty¬—from general dentistry and plastics to urology and dermatology—still don't need or can't justify the expense of retaining an anesthesiologist on staff.

This need has spurred the creation of a marketplace of mobile anesthesiologists in the United States, often working independently or in small partnerships. Mobile anesthesiology involves providing anesthesia services outside traditional hospital or surgical settings, such as in outpatient clinics, dental offices, or even patients'homes. Mobile anesthesiologists bring specialized equipment and expertise directly to the location, enabling safe and effective sedation or anesthesia for procedures. This approach enhances patient convenience, reduces costs, and increases access to care, particularly in underserved areas or for patients with mobility challenges. However, it requires careful risk management, regulatory compliance, and logistical coordination to ensure safety and efficacy in non-traditional environments.

Mobile anesthesiologists face a range of obstacles in operating their practices. Coordinating schedules, transporting equipment, and managing setup in diverse locations can be time-consuming and operationally complex. Managing payment procedures is difficult and uncoordinated. Ensuring compliance with local, state, and federal regulations can be complex, particularly concerning patient safety standards and controlled substances. In addition, there is a lack of use of Electronic Medical Records (EMR), in spite of many advantages to its use.

In the current art, existing software solutions are fragmented in nature, not tailored to the specific requirements of mobile anesthesiology, and cost-prohibitive or unreliable.

### SUMMARY

In an embodiment, a system is provided for coordinating mobile anesthesia services using a modular, case-centric architecture. The system supports scheduling, patient workup, intraoperative documentation, controlled substance compliance, billing, and related administrative workflows through interoperable modules that exchange case-associated information without centralizing clinical decision-making.

In an embodiment, the system maintains a shared case context that links information generated across multiple stages of a procedure, including appointment scheduling, patient intake, intraoperative events, compliance records, and billing records. Each module operates within its respective functional scope while accessing case-associated information generated by other modules through defined interfaces. The modular architecture allows individual components to operate independently while remaining interoperable through shared case identifiers and structured data representations.

In an embodiment, the system includes a scheduling module configured to coordinate appointment requests between clinics and mobile anesthesia providers while maintaining provider availability confidentiality. Scheduling recommendations may account for case context, predicted durations, buffer intervals, and historical outcomes, without exposing underlying provider calendars.

In an embodiment, the system includes a patient workup module that assembles a case-contextual patient profile using patient-provided intake information and synchronized electronic medical record data. The patient workup module may surface relevance indicators and risk indicators to assist provider review while preserving the provenance of underlying source records and maintaining provider authority over clinical determinations.

In an embodiment, the system includes an intraoperative module configured to support real-time documentation during a procedure. The intraoperative module may receive voice input, device-generated data, and manual inputs, and may generate structured intraoperative events following provider confirmation. Voice-based charting may be supported through speech parsing, command validation, and confirmation interfaces that ensure no intraoperative event is recorded without provider approval.

In an embodiment, the system includes a controlled substance compliance module configured to track controlled substances across acquisition, allocation, intraoperative usage, waste documentation, and audit logging. The compliance module maintains immutable, time-stamped records and supports regulatory reporting without performing clinical decision-making or adjudicating responsibility.

In an embodiment, the system includes a billing and payment module configured to manage financial workflows associated with a case, including pricing determination, deposits and prepayments, payment processing, adjustments, invoicing, and financial reporting. Billing operations are maintained separately from clinical workflows and compliance functions.

In an embodiment, the system includes an artificial intelligence and machine learning support layer that provides assistive outputs across multiple modules. The AI and machine learning support layer may generate suggestions, predictions, anomaly indicators, or draft components based on system data, while preserving human review and confirmation. Outputs generated by the AI and machine learning support layer do not determine clinical actions, compliance determinations, or financial approvals.

In an embodiment, the system preserves traceability and provenance of case-associated information across modules by maintaining audit logs, versioning, and actor attribution. The system architecture supports flexible deployment, allowing modules to be used together or independently while remaining interoperable through shared case context.

### BRIEF DESCRIPTION OF THE DRAWINGS

FIG. 1 illustrates a block diagram of system access in an embodiment.

FIG. 2 illustrates the modules of the system in an embodiment.

FIG. 3 illustrates a clinician scheduling interface in an embodiment.

FIG. 4 illustrates a provider scheduling interface showing clinics in an embodiment.

FIG. 5 illustrates a provider scheduling interface showing patients in an embodiment.

FIG. 6 illustrates an interface for a patient workup in an embodiment.

FIG. 7 illustrates a patient chart in an embodiment of the system.

FIG. 8 illustrates a payment interface in an embodiment of the system.

FIG. 9 illustrates a back office interface in an embodiment of the system.

FIG. 10 is a block diagram of the case scheduling module of FIG. 2.

FIG. 11 illustrates a functional block diagram of an AI and machine learning support layer of FIG. 2 in an embodiment of the system.

FIG. 12 illustrates a functional block diagram of a patient workup module 1200 in an embodiment of the system.

FIG. 13 illustrates a functional block diagram of an intraoperative module 203 in an embodiment of the system.

FIG. 14 illustrates a functional block diagram of a controlled substance compliance module 205 in an embodiment of the system.

FIG. 15 illustrates a functional block diagram of a billing and payment module 204 in an embodiment of the system.

FIG. 16 illustrates an example computing system in an embodiment.

### DETAILED DESCRIPTION OF THE SYSTEM

The system addresses the disadvantages of current mobile anesthesiology systems by providing a single solution that handles all aspects form the clinician side, the patient side, and the provider side. The system provides solutions in 1. Case Scheduling, 2. Patient Workup (EMR), 3. Intraoperative (Clinical), 4. Payment Solutions, and 5. Back Office.

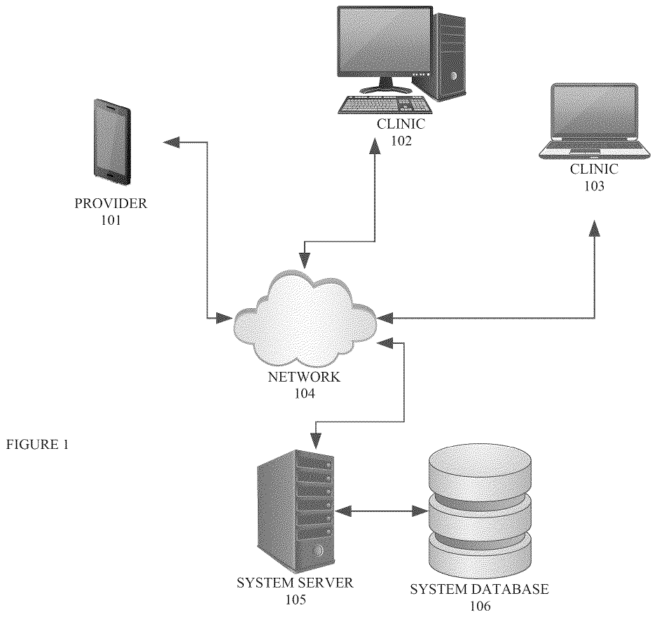

FIG. 1 illustrates the overall system in an embodiment. The system can be accessed by any computing device, including smartphones, desktop computers, tablet computers, laptop computers, and the like. A provider 101 can access the system via the cloud (e.g. Network 104, which in an embodiment is the Internet) and access the System Server 105 and System Database 106. Any number of clinics and even hospitals, such as Clinic 102 or Clinic 103, can also access the system via the Network 104. The System Server implements modules for case scheduling, patient workup, intraoperative, payment solutions, and back office.

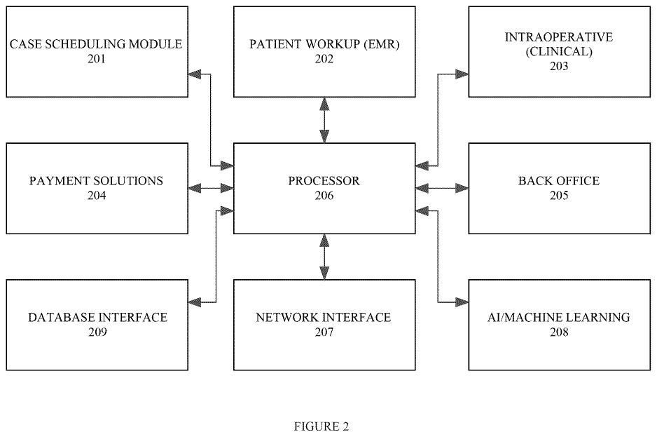

FIG. 2 illustrates a block diagram of the modules of the system in an embodiment. The system comprises a Processor 206 coupled to Case Scheduling Module 201, Patient WorkUp Module 202, Intraoperative Module 203, Payment Solutions Module 204, and Back Office Module 205. In addition, the Processor 206 is coupled to Database Interface 209, Network Interface 207, and AI/Machine Learning 208.

In an embodiment, the system operates using a case-centric data architecture in which information associated with a procedure is linked to a common case identifier and exchanged among system modules through defined interfaces. Each module generates, consumes, or annotates case-associated information within its respective functional scope without assuming control over other modules. Case-associated information may include scheduling context, patient intake data, intraoperative events, compliance records, and billing records. Modules may access case-associated information generated by other modules without duplicating underlying source records, and updates to case-associated information are recorded in a manner that preserves provenance and traceability. The modular architecture allows individual modules to operate independently while remaining interoperable through shared case context.

### Case Scheduling Module 201

Organizing appointments between mobile anesthesiologists and their referring offices is a source of potential error. Prior to the present system, the process was manual, relying on asynchronous communication with its accompanying risk of miscommunication, double-booking, and preventable cancelations. Appointment requests would be agreed over the phone or in person, but not get subsequently booked on to the clinician or provider calendar. Inadvertent double bookings were a risk, along with last-minute appointments blindsiding the anesthesiologists where the clinic simply forgot to make the request despite having arranged for their patient to come in.

The case scheduling module of the present system brings certainty to the process. All booking requests are entered directly into the proprietary system along with preliminary information about the patient. This allows the anesthesiologist to make a provisional assessment of the patient's suitability for office-based anesthesia.

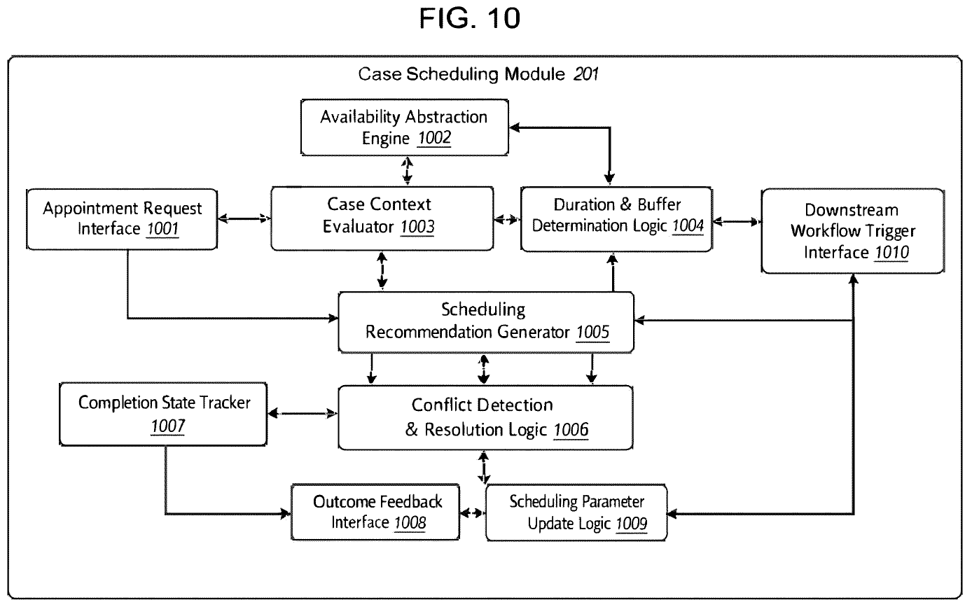

FIG. 10 illustrates a functional block diagram of the case scheduling module 201, and illustrates internal functional components used to evaluate appointment requests, generate scheduling recommendations, and update scheduling parameters based on observed outcomes.

In an embodiment, the case scheduling module 201 includes an appointment request interface 1001 configured to receive scheduling requests from clinics or offices. Appointment requests may include a proposed date or time, a procedure type, an office identifier, and preliminary patient information. The appointment request interface 1001 communicates request data to a case context evaluator 1003 for further processing.

The case scheduling module 201 further includes an availability abstraction engine 1002. The availability abstraction engine 1002 maintains a representation of provider availability without exposing a provider's full calendar to requesting offices. In an embodiment, the availability abstraction engine 1002 stores availability constraints, provider preferences, and relationship metadata in a structured format that allows feasibility evaluation while limiting disclosure of underlying scheduling details. Such a structured format may include: Temporal constraints, including permissible days of the week, time-of-day ranges, maximum case counts per day, or minimum spacing between cases; Buffer and travel constraints, including minimum setup or teardown intervals and maximum allowable travel distances or travel time between cases; Preference parameters, including preferred procedure categories, office-specific preferences, or exclusions; and Relationship metadata, including clinic-specific permissions, priority levels, or historical collaboration indicators.

The case context evaluator 1003 evaluates appointment request data using case-specific context, including procedure category, estimated complexity, provider preferences, and office-related metadata. The case context evaluator 1003 receives feasibility inputs from the availability abstraction engine 1002 and supplies contextual information to duration and buffer determination logic 1004. As used herein, feasibility inputs refers to constraint-based indicators generated by the availability abstraction engine that identify whether proposed appointment times are compatible with provider availability without exposing an underlying calendar.

The duration and buffer determination logic 1004 determines predicted case duration and one or more buffer intervals associated with a case. Buffer intervals may correspond to setup, teardown, and travel time between locations. In an embodiment, buffer intervals may vary based on provider, procedure type, office characteristics, or other observed factors. The duration and buffer determination logic 1004 supplies time constraints to a scheduling recommendation generator 1005.

The scheduling recommendation generator 1005 produces one or more candidate appointment time slots that satisfy availability constraints, buffer constraints, and case context constraints. Candidate time slots may be returned to the appointment request interface 1001 for presentation to a requesting clinic or office.

In an embodiment, the case scheduling module 201 further includes conflict detection and resolution logic 1006. The conflict detection and resolution logic 1006 evaluates proposed appointment times against existing confirmed cases and associated buffer intervals. When a conflict is detected, the conflict detection and resolution logic 1006 flags the conflict and supplies alternate scheduling recommendations to the scheduling recommendation generator 1005.

The case scheduling module 201 also includes a completion state tracker 1007. The completion state tracker 1007 monitors completeness of appointment-related information, including intake data and required forms, and generates completion indicators associated with scheduled cases. Completion state information may be presented to providers or offices to identify appointments that are pending information prior to a scheduled date.

In an embodiment, outcome data associated with completed cases is supplied to an outcome feedback interface 1008. Outcome data may include actual case duration, actual teardown time, and actual travel time. The outcome feedback interface 1008 supplies such data to scheduling parameter update logic 1009, which updates one or more scheduling parameters used by the duration and buffer determination logic 1004 for future scheduling evaluations.

The case scheduling module 201 further includes a downstream workflow trigger interface 1010. Upon acceptance of a scheduling recommendation, the downstream workflow trigger interface 1010 initiates one or more downstream system actions, including activation of patient intake workflows and coordination with other system modules.

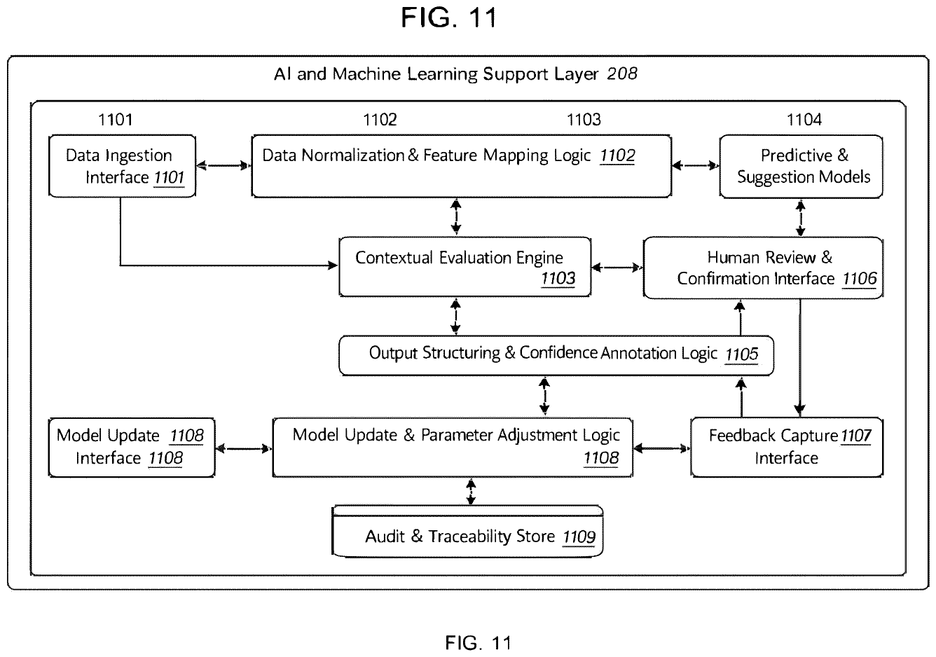

FIG. 11 illustrates a functional block diagram of an AI and machine learning support layer 208 in an embodiment of the system. The AI and machine learning support layer 208 provides system-level assistance functions that operate across multiple modules of the system without supplanting provider judgment or clinical decision-making. In an embodiment, the AI and machine learning support layer 208 processes data generated by the system to produce structured outputs that may be reviewed, confirmed, modified, or rejected by a user.

In an embodiment, the AI and machine learning support layer 208 includes a data ingestion interface 1101. The data ingestion interface 1101 receives data from one or more system modules, including scheduling data, patient intake data, electronic medical record data, intraoperative event data, billing data, and compliance-related data. The data ingestion interface 1101 may receive both structured and semi-structured data and does not alter the source records from which the data is obtained.

The AI and machine learning support layer 208 further includes data normalization and feature mapping logic 1102. The data normalization and feature mapping logic 1102 transforms ingested data into a shared internal representation suitable for further evaluation. In an embodiment, the data normalization and feature mapping logic 1102 performs one or more of field standardization, unit normalization, temporal alignment, and feature mapping. The resulting feature representations are used for subsequent analysis without generating clinical conclusions.

A contextual evaluation engine 1103 evaluates normalized data in view of case context and system state. In an embodiment, the contextual evaluation engine 1103 determines which AI-supported functions are applicable based on factors such as workflow phase, provider configuration, and case characteristics. The contextual evaluation engine 1103 routes feature data to one or more predictive or suggestion models 1104.

The predictive and suggestion models 1104 generate non-deterministic outputs based on the supplied feature data. In an embodiment, the predictive and suggestion models 1104 generate one or more of scheduling-related predictions, risk indicators, relevance suggestions for patient data, anomaly flags, or draft narrative components. Outputs generated by the predictive and suggestion models 1104 are not final system actions and do not constitute medical diagnoses or decisions.

Output structuring and confidence annotation logic 1105 converts outputs generated by the predictive and suggestion models 1104 into structured system artifacts. In an embodiment, such artifacts include suggested time windows, highlighted data elements, risk tiers, or draft text components. The output structuring and confidence annotation logic 1105 may associate outputs with contributing factors or confidence indicators to provide transparency into the basis of the generated outputs.

The AI and machine learning support layer 208 further includes a human review and confirmation interface 1106. The human review and confirmation interface 1106 presents AI-generated outputs to a user for review. In an embodiment, the user may accept, modify, or reject the outputs. No AI-generated output is finalized or acted upon by the system without user confirmation.

A feedback capture interface 1107 captures outcome information associated with reviewed outputs. In an embodiment, captured feedback includes user overrides, corrections, and post-action outcomes such as actual case duration or documented events. The feedback capture interface 1107 associates feedback with corresponding AI-generated outputs.

Model update and parameter adjustment logic 1108 updates one or more parameters of the predictive and suggestion models 1104 based on accumulated feedback. In an embodiment, model updates are performed using anonymized or de-identified data. The model update and parameter adjustment logic 1108 does not retroactively alter historical system records.

An audit and traceability store 1109 maintains records linking ingested data, AI-generated outputs, user confirmations, and subsequent outcomes. In an embodiment, the audit and traceability store 1109 supports compliance, review, and training by preserving traceable associations between inputs, outputs, and user actions.

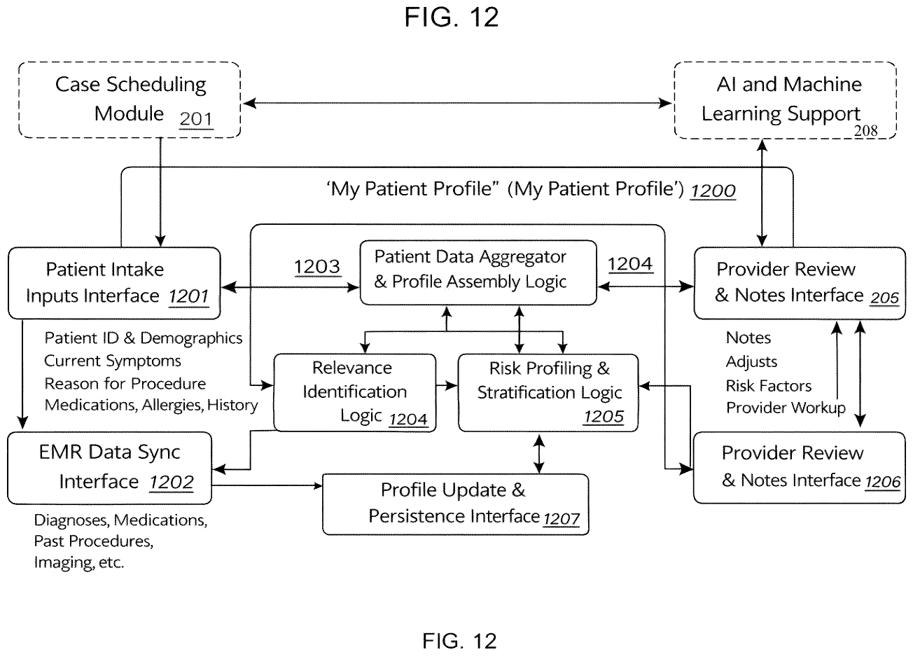

FIG. 12 illustrates a functional block diagram of a patient workup module 1200 in an embodiment of the system. The patient workup module 1200, also referred to as a “MyPatientProfile” module, provides a case-contextual patient profile assembled from multiple data sources to support anesthesia preparation and review. The patient workup module 1200 operates in coordination with other system modules while preserving the provenance and integrity of underlying source records.

In an embodiment, the patient workup module 1200 receives case context information from a case scheduling module 201. Case context information may include a procedure type, scheduled date and time, and associated clinic information. The case context information is used to scope and contextualize patient data assembled within the patient workup module 1200.

The patient workup module 1200 includes a patient intake inputs interface 1201. The patient intake inputs interface 1201 receives patient-provided and clinic-provided information, including demographic information, current symptoms, reason for the procedure, medication lists, allergy information, and medical history. Intake information may be entered directly by a patient, by clinic staff, or by a provider, and is associated with a corresponding case.

The patient workup module 1200 further includes an EMR data synchronization interface 1202. The EMR data synchronization interface 1202 receives external clinical data from one or more electronic medical record sources. Such data may include diagnoses, prior procedures, medications, laboratory results, imaging data, and other clinical records. In an embodiment, the EMR data synchronization interface 1202 maintains references to source records rather than duplicating the records themselves, thereby preserving source ownership and auditability.

Intake data received through the patient intake inputs interface 1201 and clinical data received through the EMR data synchronization interface 1202 are supplied to a patient data aggregator and profile assembly logic 1203. The patient data aggregator and profile assembly logic 1203 assembles a case-specific patient profile by associating intake data and synchronized EMR data with the relevant case context. In an embodiment, the assembled patient profile comprises a curated subset of patient data relevant to anesthesia preparation while maintaining links to the underlying source records.

In an embodiment, the patient workup module supports repeat patient recognition across multiple offices or clinical locations. The system may identify potential associations between patient records originating from different sources based on one or more attributes, including demographic information, historical procedure data, or other identifying metadata. Such associations are evaluated without automatically merging underlying records.

In an embodiment, the system generates a confidence indication associated with a proposed patient association. The confidence indication may be derived from similarity across multiple data attributes rather than from a single identifier. Proposed associations and corresponding confidence indications may be presented to a provider or authorized user for review.

In an embodiment, confirmation or rejection of a proposed patient association is recorded by the system and linked to the case context. Confirmed associations may allow patient-related information from prior cases or offices to be referenced in subsequent patient workup processes while preserving the provenance of the underlying source records.

In an embodiment, patient association events, including proposed associations, confirmations, and rejections, are recorded in an audit log with timestamp and actor attribution. The system does not overwrite or consolidate original patient records based solely on automated association, and cross-office associations remain subject to human confirmation.

The patient workup module 1200 further includes relevance identification logic 1204. The relevance identification logic 1204 evaluates assembled patient data to identify data elements that may be pertinent to the current case. In an embodiment, relevance identification logic 1204 surfaces candidate data elements for provider review without suppressing or removing other data from the underlying records. Relevance identification logic 1204 may be informed by outputs provided by an AI and machine learning support layer 208, as described with reference to FIG. 11.

The patient workup module 1200 also includes risk profiling and stratification logic 1205. The risk profiling and stratification logic 1205 evaluates patient data to generate structured risk indicators associated with the case. Risk indicators may reflect combinations of comorbidities, medications, prior anesthetic events, or other factors. The outputs of the risk profiling and stratification logic 1205 are assistive and do not constitute medical diagnoses or clinical determinations. In an embodiment, risk profiling and stratification logic 1205 may be informed by outputs provided by the AI and machine learning support layer 208.

In an embodiment, the patient workup module generates composite risk indicators associated with a case by evaluating multiple patient-related factors in combination. Composite risk indicators may reflect interactions among comorbidities, medications, prior anesthetic history, and other patient data elements rather than individual factors in isolation. Composite risk indicators are structured outputs intended to assist provider review and do not constitute clinical determinations.

In an embodiment, composite risk indicators may be represented as categorical tiers, numeric scores, or other structured representations suitable for display and downstream use. Composite risk indicators may be supplied to other system modules as part of the shared case context, including scheduling or intraoperative preparation workflows, without controlling or restricting module operation.

In an embodiment, outcome information associated with completed cases may be used to adjust parameters associated with composite risk profiling. Outcome information may include documented intraoperative events, recovery outcomes, or post-procedure annotations. Parameter adjustments may be performed using aggregated or de-identified information and do not retroactively alter prior risk indicators or historical records.

In an embodiment, composite risk profiling functions may be assisted by the artificial intelligence and machine learning support layer described with reference to FIG. 11. Outputs generated with AI assistance remain subject to provider review and confirmation and do not replace provider judgment.

The assembled patient profile, along with relevance indicators and risk indicators, is presented to a provider through a provider review and notes interface 1206. The provider review and notes interface 1206 allows a provider to review patient data, add notes, confirm or adjust relevance indications, and perform provider-directed workup actions. Provider input entered through the provider review and notes interface 1206 is authoritative with respect to the assembled patient profile.

Provider-confirmed profile information and annotations are supplied to a profile update and persistence interface 1207. The profile update and persistence interface 1207 stores provider-confirmed selections, annotations, and versioning information associated with the patient profile. In an embodiment, the profile update and persistence interface 1207 maintains a record of which data elements were reviewed or considered for a given case, thereby supporting traceability and later review.

In an embodiment, outputs generated by the patient workup module 1200 may be used by other system modules, including intraoperative documentation and post-procedure workflows, without altering the underlying source records from which patient data was obtained.

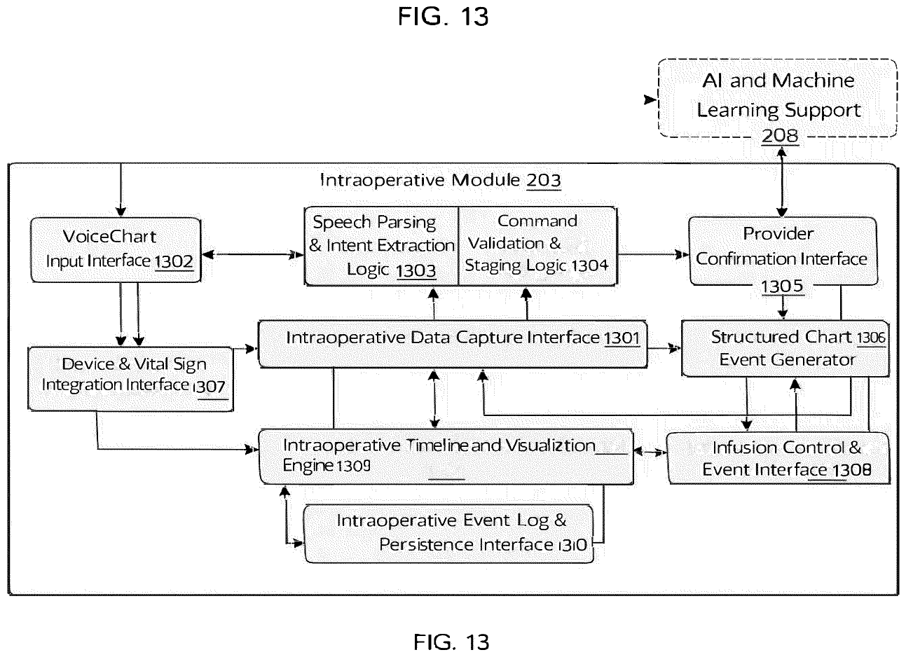

FIG. 13 illustrates a functional block diagram of an intraoperative module 203 in an embodiment of the system. The intraoperative module 203 supports real-time documentation and event capture during an anesthesia case, including voice-based charting, device-generated data ingestion, and structured event generation. The intraoperative module 203 operates during an active procedure and produces a time-aligned intraoperative record without supplanting provider judgment or clinical decision-making.

In an embodiment, the intraoperative module 203 includes an intraoperative data capture interface 1301. The intraoperative data capture interface 1301 serves as a central aggregation point for intraoperative inputs, including voice-generated inputs, device-generated data, and manually entered information. Incoming inputs are normalized and associated with a case timeline for further processing and visualization.

The intraoperative module 203 further includes a VoiceChart input interface 1302. The VoiceChart input interface 1302 receives spoken input from a provider during an active case. Spoken input may include charting commands, procedural annotations, medication-related events, or navigation instructions. The VoiceChart input interface 1302 forwards received speech data for interpretation without directly modifying the intraoperative record.

Speech received through the VoiceChart input interface 1302 is supplied to speech parsing and intent extraction logic 1303. The speech parsing and intent extraction logic 1303 converts spoken input into structured intents and associated parameters based on contextual information, including the phase of the case and previously recorded events.

Parsed intents generated by the speech parsing and intent extraction logic 1303 are evaluated by command validation and staging logic 1304. The command validation and staging logic 1304 validates proposed commands against the current case state and stages commands for review prior to execution or recording. In an embodiment, commands are not committed to the intraoperative record until confirmation is obtained.

Staged commands are presented to a provider through a provider confirmation interface 1305. The provider confirmation interface 1305 allows a provider to accept, modify, or reject staged commands. Provider confirmation is authoritative, and no intraoperative chart event is generated without provider approval.

Upon provider confirmation, confirmed commands are supplied to a structured chart event generator 1306. The structured chart event generator 1306 generates timestamped, structured intraoperative chart events corresponding to confirmed commands. Generated chart events are supplied to the intraoperative data capture interface 1301 for incorporation into the intraoperative record.

The intraoperative module 203 also includes a device and vital sign integration interface 1307. The device and vital sign integration interface 1307 receives data streams from one or more intraoperative monitoring devices, including physiologic monitors and related equipment. Device-generated data is associated with the case timeline and supplied to the intraoperative data capture interface 1301.

An infusion control and event interface 1308 provides an interface for documenting infusion-related events during a case. In an embodiment, the infusion control and event interface 1308 records infusion start, stop, pause, and rate adjustment events, which are supplied to the intraoperative data capture interface 1301 for timeline association.

The intraoperative module 203 further includes an intraoperative timeline and visualization engine 1309. The intraoperative timeline and visualization engine 1309 presents a synchronized visual representation of intraoperative events captured through the intraoperative data capture interface 1301. The timeline may include charted events, device-generated data, and infusion events, enabling rapid review during and after a procedure.

In an embodiment, one or more components of the intraoperative module 203 may be assisted by an AI and machine learning support layer 208, as described with reference to FIG. 11. The AI and machine learning support layer 208 may provide assistive outputs for speech interpretation or command validation without determining clinical actions or final chart entries.

In an embodiment, the intraoperative module 203 includes an intraoperative event log and persistence interface 1310. The intraoperative event log and persistence interface 1310 stores intraoperative information associated with a case, including timestamped chart events generated by the structured chart event generator 1306, device-generated data received through the device and vital sign integration interface 1307, and infusion-related events recorded through the infusion control and event interface 1308. In an embodiment, the intraoperative event log and persistence interface 1310 maintains source attribution indicating whether a stored event originated from a voice-confirmed entry, a device-derived data stream, or a manual entry. The intraoperative event log and persistence interface 1310 may provide persisted intraoperative data for later retrieval, review, and downstream workflows.

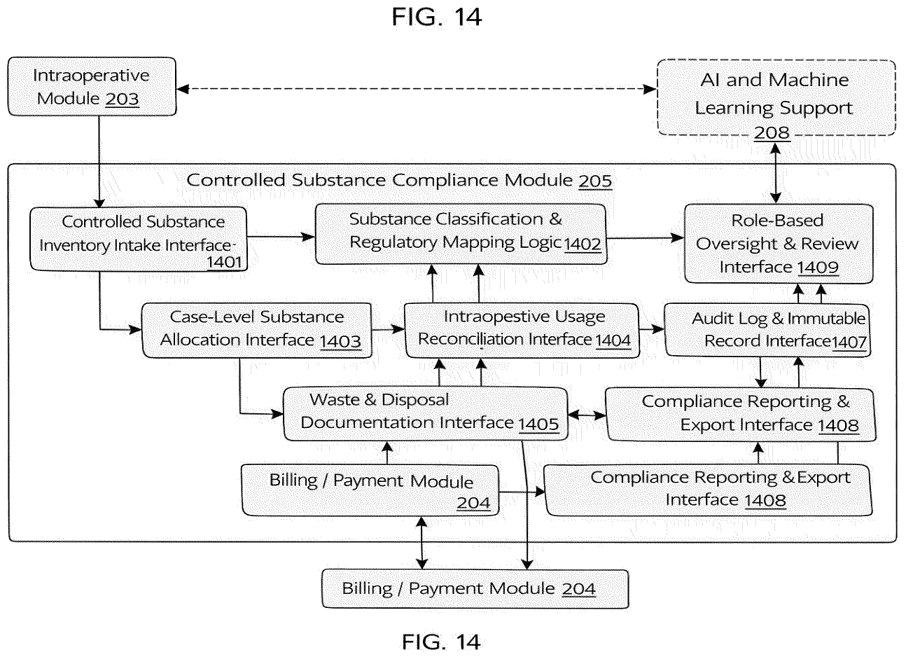

FIG. 14 illustrates a functional block diagram of a controlled substance compliance module 205 in an embodiment of the system. The controlled substance compliance module 205 supports inventory management, usage reconciliation, waste documentation, audit logging, and regulatory reporting associated with controlled substances used during clinical procedures. The controlled substance compliance module 205 operates independently of clinical decision-making while maintaining traceable associations between inventory events, case usage, and compliance records.

In an embodiment, the controlled substance compliance module 205 includes a controlled substance inventory intake interface 1401. The controlled substance inventory intake interface 1401 records acquisition events associated with controlled substances, including receipt of substances from authorized suppliers. Inventory intake information may include substance identifiers, lot or batch identifiers, quantities, and receipt dates. Inventory intake events establish an inventory baseline for subsequent allocation and reconciliation.

The controlled substance compliance module 205 further includes substance classification and regulatory mapping logic 1402. The substance classification and regulatory mapping logic 1402 associates controlled substances with corresponding classification information and regulatory attributes. In an embodiment, regulatory attributes may reflect jurisdiction-specific requirements applicable to inventory tracking, reconciliation, and reporting. The substance classification and regulatory mapping logic 1402 does not determine clinical use or dosing of substances.

Inventory units recorded through the controlled substance inventory intake interface 1401 may be associated with individual cases using a case-level substance allocation interface 1403. The case-level substance allocation interface 1403 associates one or more inventory units with a specific procedure, provider, or case identifier, enabling case-specific tracking of controlled substance usage.

The controlled substance compliance module 205 further includes an intraoperative usage reconciliation interface 1404. The intraoperative usage reconciliation interface 1404 receives usage information associated with administered substances from an intraoperative module 203. In an embodiment, the intraoperative usage reconciliation interface 1404 compares administered amounts to previously allocated inventory units to identify discrepancies or variances between allocated, administered, and remaining quantities. The intraoperative usage reconciliation interface 1404 records reconciliation results without adjudicating responsibility or determining corrective actions.

The controlled substance compliance module 205 also includes a waste and disposal documentation interface 1405. The waste and disposal documentation interface 1405 records waste events associated with controlled substances, including quantities disposed, disposal methods, and witness confirmations when applicable. Waste documentation events are associated with corresponding inventory units and cases to maintain a complete chain-of-custody record.

Inventory balances are updated using an inventory balance and adjustment logic 1406. The inventory balance and adjustment logic 1406 reflects changes to inventory quantities based on intake events, administered usage, documented waste, and authorized adjustments. In an embodiment, inventory balance updates preserve historical inventory states and do not overwrite prior records.

The controlled substance compliance module 205 further includes an audit log and immutable record interface 1407. The audit log and immutable record interface 1407 maintains time-stamped records of inventory intake, allocation, usage reconciliation, waste documentation, and adjustments. Records stored through the audit log and immutable record interface 1407 include actor attribution and event provenance to support compliance review and regulatory audits.

A compliance reporting and export interface 1408 generates reports based on records maintained by the controlled substance compliance module 205. In an embodiment, reports generated through the compliance reporting and export interface 1408 are formatted for submission to regulatory agencies, internal audit teams, or other authorized reviewers. Report generation does not modify underlying compliance records.

The controlled substance compliance module 205 further includes a role-based oversight and review interface 1409. The role-based oversight and review interface 1409 allows authorized users to review compliance records, audit logs, reconciliation results, and reports based on assigned permissions. In an embodiment, the role-based oversight and review interface 1409 supports annotation and review workflows without altering original event records.

In an embodiment, one or more functions of the controlled substance compliance module 205 may be assisted by an AI and machine learning support layer 208, as described with reference to FIG. 11. The AI and machine learning support layer 208 may provide assistive outputs such as anomaly indicators or reconciliation suggestions. Outputs generated by the AI and machine learning support layer 208 do not create compliance determinations or replace human review.

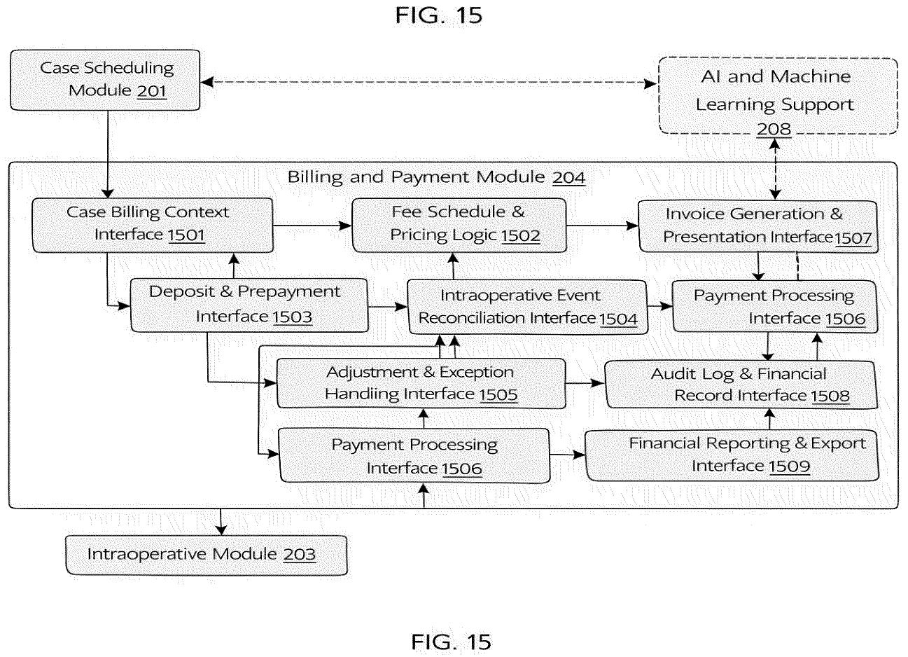

FIG. 15 illustrates a functional block diagram of a billing and payment module 204 in an embodiment of the system. The billing and payment module 204 manages financial workflows associated with scheduled and completed cases, including pricing determination, deposits and prepayments, payment processing, adjustments, and financial recordkeeping. The billing and payment module 204 operates independently of clinical decision-making and does not determine medical necessity, diagnosis, or insurance eligibility.

In an embodiment, the billing and payment module 204 includes a case billing context interface 1501. The case billing context interface 1501 receives case-related information from a case scheduling module 201 and, in some embodiments, from an intraoperative module 203. Case-related information may include a case identifier, scheduled services, provider identifiers, clinic identifiers, and timing information. The case billing context interface 1501 establishes a billing context associated with a specific case without performing pricing determinations.

The billing and payment module 204 further includes a fee schedule and pricing logic 1502. The fee schedule and pricing logic 1502 determines applicable fees for a case based on the established billing context and stored pricing configurations. In an embodiment, pricing configurations may be associated with provider agreements, clinic agreements, or service categories. The fee schedule and pricing logic 1502 does not determine insurance coverage or adjudicate claims.

In an embodiment, the billing and payment module 204 includes a deposit and prepayment interface 1503. The deposit and prepayment interface 1503 supports collection and association of deposits or advance payments with a case prior to performance of services. Deposits and prepayments may be recorded against the billing context and later reconciled with final charges.

The billing and payment module 204 also includes an intraoperative event reconciliation interface 1504. The intraoperative event reconciliation interface 1504 receives event-related information from the intraoperative module 203, such as time-based data or documented service events. In an embodiment, the intraoperative event reconciliation interface 1504 compares estimated services reflected in the billing context with actual intraoperative events to support reconciliation of charges.

Adjustments and exceptions are handled through an adjustment and exception handling interface 1505. The adjustment and exception handling interface 1505 records adjustments, credits, or manual modifications applied to a case. In an embodiment, original pricing and payment records are preserved, and adjustments are recorded as separate events to maintain financial traceability.

The billing and payment module 204 further includes a payment processing interface 1506. The payment processing interface 1506 interfaces with one or more external payment processing systems to perform payment authorization, capture, refunds, or reversals. In an embodiment, the payment processing interface 1506 does not store raw payment credentials and relies on external payment processors for secure transaction handling.

Invoices are generated using an invoice generation and presentation interface 1507. The invoice generation and presentation interface 1507 generates invoices associated with a case for presentation to a patient, clinic, or other authorized party. Invoices may reflect deposits, final charges, and adjustments associated with the billing context.

Financial events associated with billing and payment activities are recorded using an audit log and financial record interface 1508. The audit log and financial record interface 1508 maintains time-stamped records of pricing determinations, payment events, adjustments, and invoice generation. Records stored through the audit log and financial record interface 1508 include actor attribution to support audit and review.

The billing and payment module 204 also includes a financial reporting and export interface 1509. The financial reporting and export interface 1509 generates financial reports for accounting, reconciliation, or internal review purposes. Report generation does not modify underlying financial records.

In an embodiment, one or more functions of the billing and payment module 204 may be assisted by an AI and machine learning support layer 208, as described with reference to FIG. 11. The AI and machine learning support layer 208 may provide assistive outputs such as anomaly indicators or pattern analysis related to billing events. Outputs generated by the AI and machine learning support layer 208 do not determine charges, approve payments, or replace human oversight.

FIG. 3 illustrates an embodiment of the Clinic scheduling interface of the system. The Dashboard 300 includes a menu region 301 to select different interfaces. A Calendar 302 allows the clinician to select a month and day and the region 303 allows the clinician to select a time of day. The calendar is coordinated through the System Server so that only dates and times that are available will be shown on the calendar, preventing double booking.

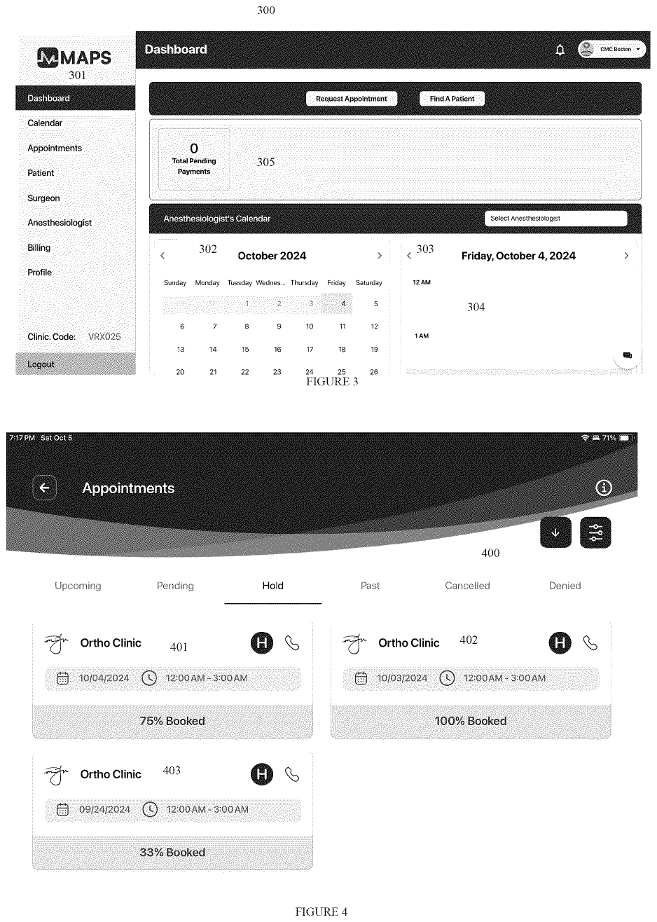

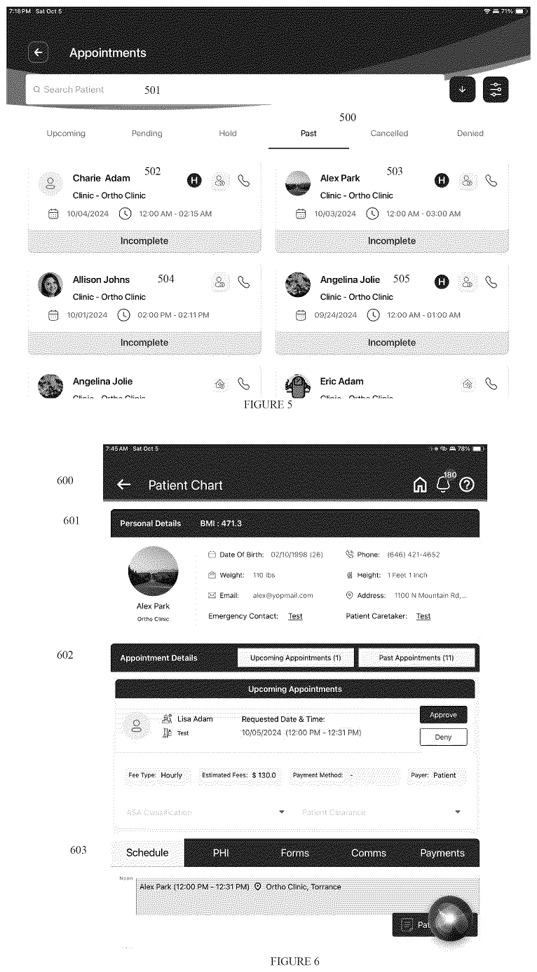

FIG. 4 illustrates an interface for the providers to review appointments on the system. The interface 400 allows the provider to select a particular clinic (e.g. Ortho Clinic in the example) and see the booking dates for that clinic. In this example there are three different date interfaces 401 (10/4) 402 (10/3) and 403 (09/24) presented. Each interface shows the time of the appointment as well as if the booking is complete. In this case, only booking 402 is complete. Booking 401 is 75% complete and booking 403 is 33% complete.

All booking requests are entered directly into the proprietary system along with preliminary information about the patient. This allows the anesthesiologist to make a provisional assessment of the patient's suitability for office-based anesthesia. Once an Appointment Request is opened, the office is prompted to input a range of demographic information about the patient. Other supporting details, such as the type of procedure and the proposed time and date of the appointment (based on the anesthesiologist's calendar availability), are entered at the same time.

In addition to reducing friction, this appointment booking process prompts clinics to think more fully through their requests before submitting them to the anesthesiologist: How much time will this patient realistically need for their procedure? Could the office schedule additional appointments for that day to get the most out of the anesthesiologist's time? (This is the basis of a Hold Appointment request: a block of time reserved to the clinic against which it can book multiple cases.) Additionally, connecting the patient with the anesthesiologist early in the process—through the sharing of the Anesthesia Intake Forms and supporting communications—helps decrease the risk of an appointment cancellation once the request has been approved. After the request is accepted by the anesthesiologist, patients are immediately brought into the process and a channel of communication between them and their anesthesiologist is established.

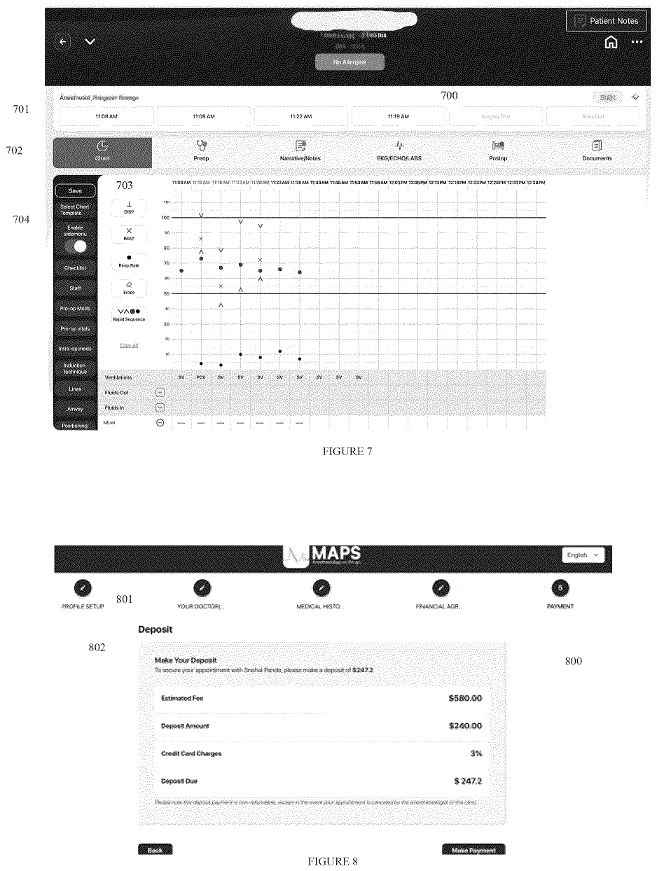

FIG. 5 illustrates a provider scheduling interface showing patients in an embodiment. The interface 500 includes a search bar 501 for looking up individual patients. Regions 502, 503, 504, and 505 display information for individual patients. The information includes upcoming appointments as well as a status as to the completeness of their records and information needed for the appointment. This allows the provider to quickly see where follow up is needed in advance of any appointments.

### Patient WorkUp Module (EMR)

In an embodiment, patient medical data is drawn from a range of information sources, including:
Qualified Health Information Networks or QHINs (created by Congress under HITECH Act 2009 and further bolstered by the 21st Century Cures Act 2016).
PCP-supplied data
Anesthesiologist requested lab work and other tests
Patient's self-attested medical history
Clinic-supplied data

This variety of medical information is important not only to ascertain the suitability of the patient for office-based anesthesia (by assigning the appropriate American Society of Anesthesiologists (“ASA”) status. The ASA Status is a risk-stratifying system used mainly by anesthesiologists to help predict preoperative risks. The system is used to assess a patient's preoperative comorbid conditions and assigns a class ranging from 1-6. The classification system is used as an additional tool with other variables such as type of surgery, frailty, and level of deconditioning in predicting perioperative risks. It also allows the anesthesiologist to build a more rounded picture of the patient before meeting them. This can help guide clinical decision-making, all the way down to the supplies—from tubing to medications—the mobile anesthesiologist packs into their vehicle before heading out to the clinic.

In an embodiment, Patient EMR is automatically assembled in the system with the anesthesiologist's ease of access in mind. FIG. 6 illustrates a patient chart in an embodiment. The Patient Chart 600 includes a personal details section 601 where important physical information about the patient is presented efficiently. This information is important and critical to determine the appropriate anesthesia protocol for the patient.

Region 602 includes appointment details for the patient, along with fee information and other administrative information. Region 603 includes a number of tabs that the provider can select from to find useful information about the patient, the procedure, payment, communications, and scheduling.

### Intraoperative (Clinical)

The Intraoperative Module 203 allows the provider to be paperless and to seamlessly populate clinical data points, from a patient's vital signs to a record of the drugs administered by amount and by method, into the anesthesia record. The ability to activate auto vital streaming (through a partner program, such as Neximatic™) removes further constraints from the anesthesiologist, freeing up time to focus on patient care.

Taken together, this digital embedding of clinical charting into a practice management solution improves performance for mobile anesthesia. It avoids the need to use an inadequate patchwork of existing tools.

The system also implements voice-controlled clinician data input for anesthesia charting. The anesthesiologist wears an earpiece and communicates verbally to the system software, detailing all salient details of the procedure as the case unfolds in real time.

This information may range from the drugs they're administering (and at what rate) to changes in the positioning of the patient's body during the procedure. It encompasses all typical details accounted for in an anesthesia chart and is intended to supplement any auto vitals already streaming into the chart from the monitors.

Speech data flows directly into the anesthesia chart and auto-populates in the correct location. This functionality allows the anesthesiologist to stay attentive to patient care rather than worrying about manual data entry while the patient is under anesthesia. This method also helps increase the accuracy and reliability of the chart's information through real-time data capture. When the anesthesiologist eventually has a chance to review the digital chart, they're able to evaluate the fullness and accuracy of the auto-populated information, updating it where required.

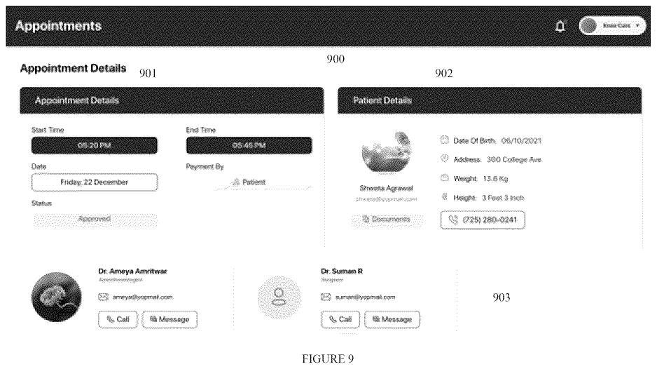

FIG. 7 illustrates a patient chart 700 in an embodiment of the system. Region 701 displays the critical Anesthesia/Surgical Timings. Region 702 allows the provider to choose from Chart (activated in this example), Preop, Narrative/Notes, EKG/ECHO/Labs, Postop, and documents. Region 703 displays information related to the selected tab. In this example, the system illustrates DBP, IMAP, Resp. Rate, Rapid Sequence, and the like. The provider can easily see time based information at a glance, with no time wasted trying to find critical information. Region 704 provides additional display choices for whichever tab is selected in region 702. When implemented with a touch based display, the provider has easy access to important patient information before, during, and after a procedure.

### Payment Solutions

The System automates the handling of payments for mobile anesthesiologists. In the prior art, when dealing with private (non-insurance) cases, solo practitioners would typically need to manually gather payment details from the patient before the appointment.

With custom-built payment functionality integrated throughout the present system payment platform, patients receive an estimated cost of service and are required to sign a financial consent form as part of the intake process. They are also asked to make a deposit, assuming the anesthesiologist has kept that feature activated. Requiring a deposit helps reduce the potential for last-minute patient cancellations for non-health-related reasons.

FIG. 8 illustrates a payment interface 800 in an embodiment of the system. Region 801 includes a number of types of information about the patient, including financial agreement to be signed by the patient, and a path for providing a deposit in advance of the procedure. The region 802 shows the estimated fee for the service, a deposit amount, and a Pay button to allow one or more methods of payment of the deposit and/or bill, including credit card, ACH, e-check, and the like.

### Back Office

Some mobile anesthesiologists hire an assistant to deal with the administration of their practice; others do the clerical work themselves. In either scenario, the administration needs to be handled.

The back office module of the present system is designed to absorb anywhere up to 90% of a mobile practitioner's back-office responsibilities. A significant chunk of this work—scheduling and payment, for example—is addressed through the functionality described above.

The system is also configured to track mileage (a mobile anesthesiologist is essentially a business on wheels), document patient communications, handle e-prescribing, and maintain a controlled drug log. Plus, anesthesia consents are captured from the patients ahead of each procedure, and securely stored digitally with zero administrative effort.

The ease of the scheduling function not only reduces uncertainty between patient, office, and anesthesiologist. It also significantly decreases the number of incoming calls to the anesthesiologist and their back-office function.

This focus on back-office automation reduces administrative efforsts an anesthesiologis, freeing up more time for actual medical work. Most importantly, it frees up the anesthesiologist's time to focus on the most important element of all: the safety and comfort of the patient throughout the process. This is particularly significant for a patient population that may already be experiencing higher levels of anxiety and unease about their upcoming procedure.

FIG. 9 illustrates a Back Office interface 900 in an embodiment of the system. The interface includes region 901 with appointment details for the patient, identifying who will be paying, whether the patient is approved, and other information. Region 902 includes some personal data about the patient, including a button to access documents, e.g., consent, reports, authorizations, and the like, that are required of the patient. This document can be autofilled by the system using information from scheduling, EMR, Intraoperative, and the like, to reduce the administrative burden on the provider and their office staff.

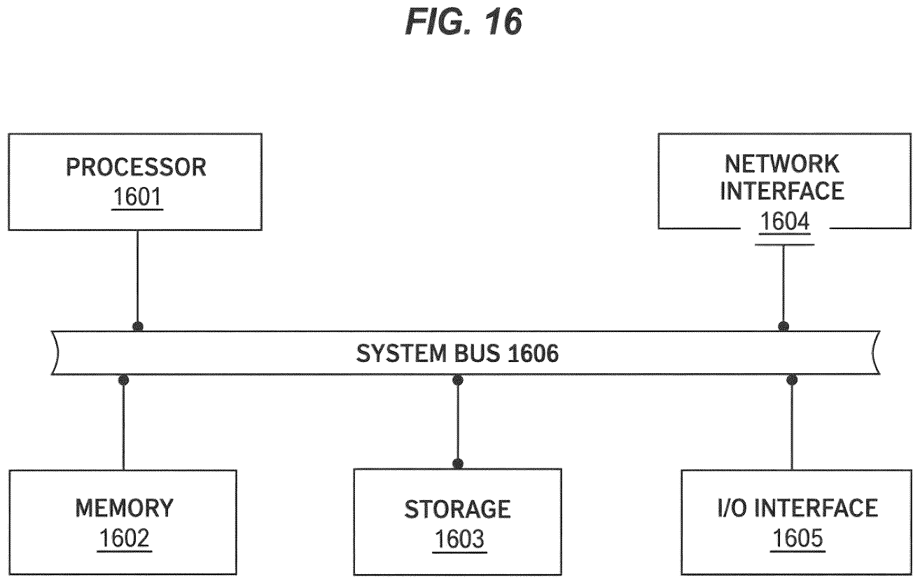

FIG. 16 illustrates an example computing system 1600 that may be used to implement one or more modules or interfaces described in this specification. In an embodiment, the computing system 1600 includes a processor 1601 configured to execute instructions stored on one or more computer-readable media. The computing system 1600 further includes memory 1602, which may store instructions and runtime data used during execution, and storage 1603, which may store data structures, event records, case-associated information, audit records, and other information in a non-volatile manner.

In an embodiment, the computing system 1600 includes a network interface 1604 configured to communicate with external systems or devices over one or more networks, and an input/output (I/O) interface 1605 configured to communicate with user interface devices and other peripherals. The processor 1601, memory 1602, storage 1603, network interface 1604, and I/O interface 1605 are communicatively coupled via a system bus 1606 or other interconnect.

In an embodiment, the computing system 1600 may be implemented using one or more distributed computing devices, servers, or cloud-based resources, and the functional modules described herein may be implemented as software components, services, or processes executing on one or more computing systems. The computing system 1600 is provided as an example and is not intended to limit the manner in which the disclosed system may be implemented.

Thus, an improved method and apparatus for managing mobile anesthesiology has been described.

What is claimed is:

1. A system for coordinating mobile anesthesia services, comprising:
one or more processors; and
one or more non-transitory computer-readable media storing instructions that, when executed by the one or more processors, cause the system to:
maintain a case-centric data structure associated with a procedure, the case-centric data structure linking scheduling information, patient workup information, intraoperative events, compliance records, and billing records;
coordinate appointment scheduling using a scheduling module configured to generate scheduling recommendations based on case context while abstracting provider availability;
assemble a case-contextual patient profile using patient intake information and electronic medical record data while preserving provenance of underlying source records;
receive intraoperative inputs including voice-generated inputs and device-generated data during performance of the procedure;
stage intraoperative inputs for provider confirmation prior to recording;
generate structured intraoperative events only after provider confirmation of staged inputs;
track controlled substances associated with the procedure across inventory intake, intraoperative usage, and waste documentation; and
manage billing and payment records associated with the procedure using case-associated information,
wherein the scheduling module, patient workup module, intraoperative module, compliance module, and billing module operate as interoperable modules linked by the case-centric data structure without centralizing clinical decision-making.

2. The system of claim 1, wherein the scheduling module generates scheduling recommendations using predicted procedure duration and buffer intervals derived from historical case outcomes.

3. The system of claim 1, wherein the scheduling module maintains provider availability using an availability abstraction that does not expose an underlying provider calendar.

4. The system of claim 1, wherein assembling the case-contextual patient profile includes identifying candidate patient records from multiple offices and generating a confidence indication associated with a proposed patient association.

5. The system of claim 4, wherein confirmation or rejection of the proposed patient association is recorded with timestamp and actor attribution.

6. The system of claim 1, wherein generating structured intraoperative events includes converting provider-confirmed voice inputs into timestamped chart events.

7. The system of claim 1, wherein the intraoperative module receives streaming physiologic data from one or more monitoring devices and associates the streaming data with the case-centric data structure.

8. The system of claim 1, wherein the compliance module maintains immutable audit records associated with controlled substance intake, usage, and waste events.

9. The system of claim 1, wherein the billing module supports collection of deposits or prepayments associated with the case prior to performance of the procedure.

10. The system of claim 1, further comprising an artificial intelligence and machine learning support layer configured to generate assistive outputs for one or more modules while preserving provider confirmation of recorded events.

11. A method for coordinating mobile anesthesia services using a computing system, comprising:
associating scheduling information, patient intake information, intraoperative events, compliance records, and billing records with a common case identifier;
generating scheduling recommendations based on case context while limiting exposure of provider availability data;
assembling a patient profile for a case using intake data and electronic medical record data while maintaining source record provenance;
receiving voice-generated input during an anesthesia procedure;
parsing the voice-generated input into structured intents;
staging the structured intents for provider confirmation prior to recording intraoperative events;
recording structured intraoperative events only after provider confirmation;
reconciling controlled substance inventory based on intraoperative usage and waste documentation; and
generating billing records associated with the case based on recorded events,
wherein the steps are performed by interoperable modules of the computing system linked by the common case identifier.

12. The method of claim 11, wherein generating scheduling recommendations includes inserting buffer intervals between scheduled procedures based on predicted teardown and travel time.

13. The method of claim 11, wherein assembling the patient profile includes generating composite risk indicators based on interactions among multiple patient-related factors.

14. The method of claim 13, wherein the composite risk indicators are adjusted based on outcomes of prior cases without altering historical records.

15. The method of claim 11, wherein staging the structured intents includes validating the intents against a current case state prior to provider confirmation.

16. The method of claim 11, wherein reconciling controlled substance inventory includes associating administered quantities and waste documentation with specific inventory units.

17. The method of claim 11, wherein generating billing records includes reconciling estimated services with intraoperative event data.

18. The method of claim 11, further comprising generating audit records linking recorded events to actor attribution and timestamps.
